(19) United States
(12) Patent Application Publication
(10) Pub. No.: US 2010/0218769 A1
(43) Pub. Date: Sep. 2, 2010

(54) RESPIRATORY ASSISTANCE DEVICE

(76) Inventor: Georges Boussignac, Antony (FR)

Correspondence Address: J. Rodman Steele;Novak Druce & Quigg LLP, 525 Okeechobee Blvd, Suite 1500, West Palm Beach, FL, 33401, US

(21) Appl. No.: 12/680,972

(22) PCT Filed: Sep. 30, 2008
(86) PCT No.: PCT/FR08/01360

§ 371 Date: Mar. 31, 2010

(30) Foreign Application Priority Data
Oct. 8, 2007 (FR) 07 07036

(51) Int. Cl. A61M 16/04

(52) U.S. Cl. 128/207.15

(57) ABSTRACT

The invention relates to a respiratory device (1) that comprises, at the distal end (3) thereof that receives gas jets breathable through channels (8) and a deflection means (14b), a sealing means (21) capable of ensuring, at the pharynx, a sealed gaseous communication with the trachea of a patient.

The present invention relates to a respiratory assistance device that can advantageously be used as an artificial respiration device during resuscitation of a person in a state of cardiac arrest.

A tubular respiratory assistance device for patients with spontaneous but insufficient breathing is already known in particular from patents U.S. Pat. No. 6,363,935, U.S. Pat. No. 6,761,172 and U.S. Pat. No. 6,814,075. A known respiratory assistance device of this kind comprises:
a tubular element which forms a main channel and which is designed to be connected via its distal end to an airway of a patient, while the proximal end of said tubular element is situated outside said patient, and the respiratory system of said patient is connected to the outside by way of said main channel;
peripheral auxiliary channels that open into said main channel, said auxiliary channels being supplied with respiratory gas; and
deflection means by which the jets of respiratory gas injected through said auxiliary channels can be made to converge on each other inside said main channel.

Thus, the patient is ventilated by said jets of respiratory gas.

In a particular embodiment, a tubular respiratory assistance device of this kind is designed to be able to be introduced via its distal end into the patient's mouth and to be pushed forward until said distal end is situated near the lungs, at the level of the carina, said respiratory assistance device thus extending through the larynx and the trachea.

It will be appreciated that placing the respiratory assistance device in the airways of a patient causes the latter discomfort and is an awkward procedure that can only be carried out by a highly qualified operator. Moreover, it can sometimes happen that said respiratory assistance device, once placed in the trachea of a conscious patient, cannot be tolerated by the latter.

Moreover, experience has shown that this respiratory assistance device for a patient with spontaneous respiration can be used successfully as an artificial respiration device (and not just as a respiratory assistance device) on persons in a state of cardiac arrest who are being resuscitated by alternate compression and decompression of the thoracic cage, the jets of said respiratory gas promoting the restoration of inhalation and the circulation of blood.

However, it will be noted that the aforementioned difficulty of placing this respiratory assistance device in the trachea of a patient in a state of cardiac arrest, combined with the agitation experienced even by a qualified operator when placing the device in the trachea in an emergency situation, may result in a loss of time that proves fatal for the patient.

The object of the present invention is therefore to improve this known device, mentioned above, in order to make it easier to put in place.

To this end, according to the invention, the respiratory assistance device which is tubular and which forms a main channel connecting the proximal end and the distal end of said device and of which said distal end is designed to be introduced into the mouth of a patient in the direction of the patient's lungs, while said proximal end is designed to remain outside the mouth of said patient, said device comprising:
at least one auxiliary channel which opens into said main channel near said distal end and through which a jet of breathable gas can be injected into said main channel; and
deflection means by which said jet of breathable gas injected through said auxiliary channel is deflected into said main channel,

is characterized in that it comprises sealing means which surround said distal end and which, in the area of the pharynx of said patient, are able to ensure an at least substantially leaktight gaseous communication between the trachea of said patient and said main channel of the device.

Thus, by virtue of the present invention, it suffices to introduce said distal end as far as the pharynx (and no longer as far as the carina by way of the larynx and the trachea) in order to provide the patient with respiratory assistance, which procedure is much easier and much quicker.

Said sealing means preferably comprise at least one cuff of annular shape which is able to surround the larynx of the patient and is inflatable under the action of an inflation gas delivered to said device. Thus, said inflatable cuff can easily be introduced in the deflated state into the mouth and the pharynx of the patient and then inflated once in place. Said inflation gas is preferably delivered to said cuff by way of at least one channel formed within the thickness of said device.

To avoid injuring the mucous membranes of the pharynx when said cuff is inflated, it is advantageous for said sealing means to comprise two cuffs of annular shape, of which one is arranged inside the other and which are able to surround the larynx of said patient, one of said cuffs being inflatable under the action of said inflation gas delivered to said device, and the other being in gaseous communication with said distal end of the device downstream of said deflection means.

Thus, said inflation gas can be at a pressure lower than that needed for good sealing, the additional pressure for the sealing being provided by the jet of breathable gas by way of said gaseous communication. The result of this is that the sealing pressure (causing trauma) is applied only during the injections of breathable gas, whereas outside the periods of these injections the pressure exerted on the pharynx by said sealing means can low enough not to cause any trauma.

Said cuff in gaseous communication with the distal end of the device can be arranged inside or outside said cuff which can be inflated by said inflation gas.

Advantageously, said sealing means are carried at the widened and slanted periphery of a trumpet-shaped component that can be attached, for example with a form fit, to said distal end of the respiratory assistance device. The fixing of said component on the latter can be temporary or permanent.

The figures in the attached drawing will show clearly how the invention can be realized. In these figures, identical reference signs designate similar elements.

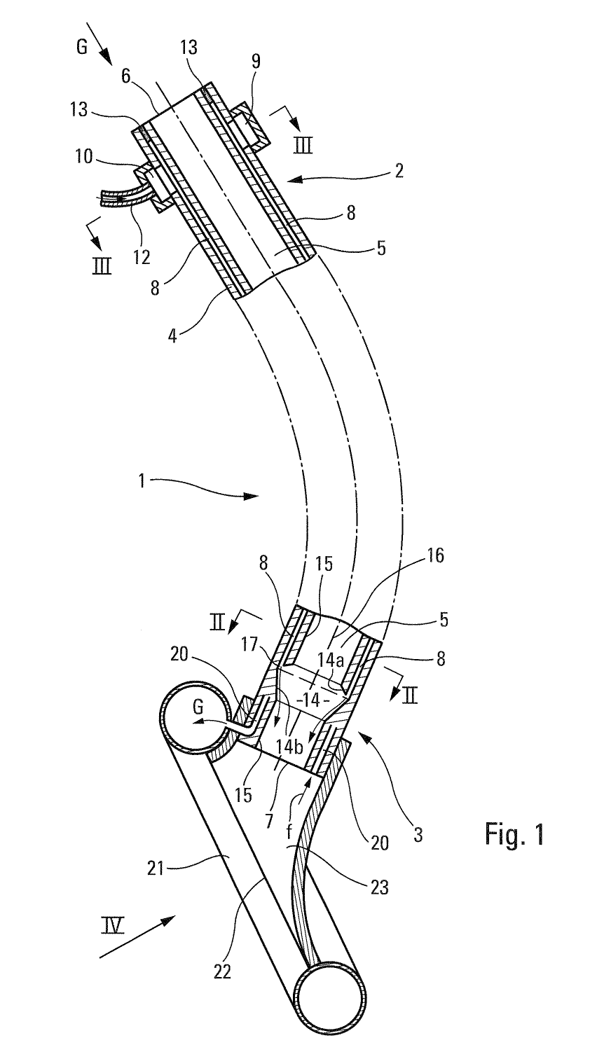

FIG. 1 is a schematic and partial view, in axial section, of an embodiment of the device according to the invention.

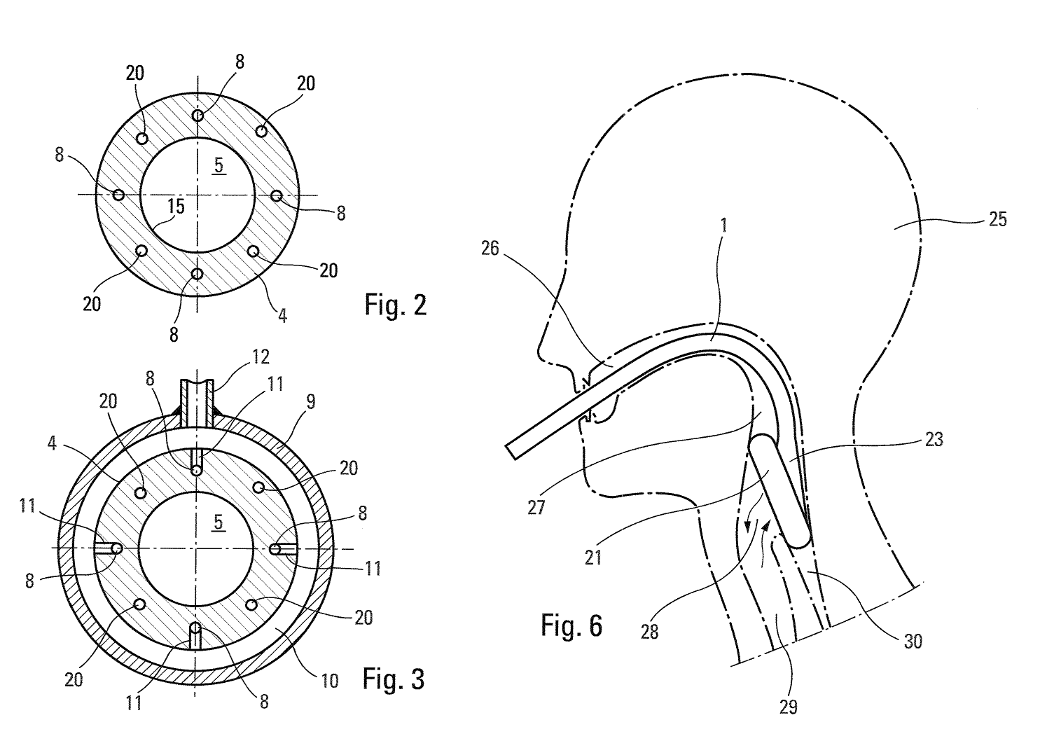

FIGS. 2 and 3 are transverse sections, respectively along lines II-II and marked in FIGS. 1, 7 and 8.

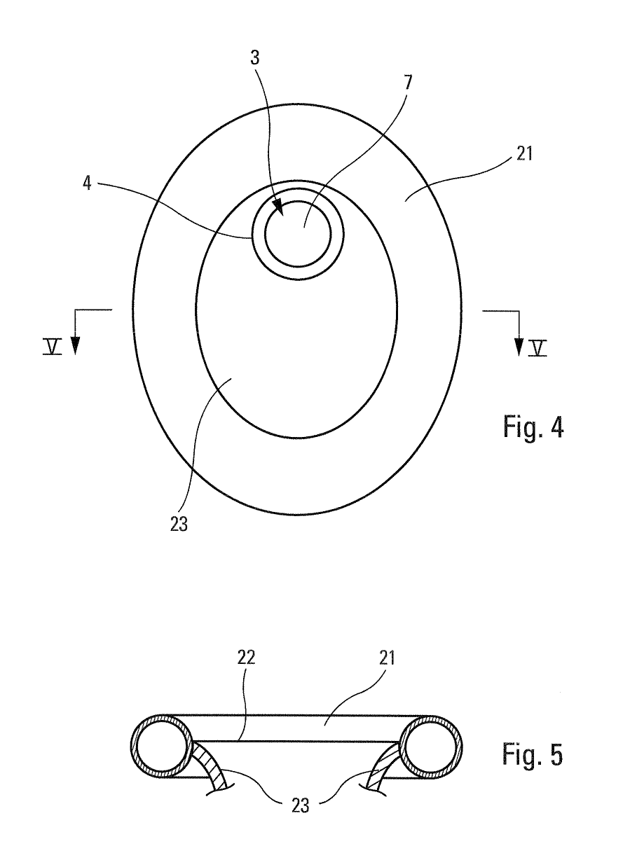

FIG. 4 is a schematic view of the sealing means of the device according to the invention, seen in the direction of the arrow IV marked in FIG. 1.

FIG. 5 is a schematic transverse section through said sealing means along line V-V marked in FIG. 4.

FIG. 6 is a schematic illustration of the placement of the device according to the invention.

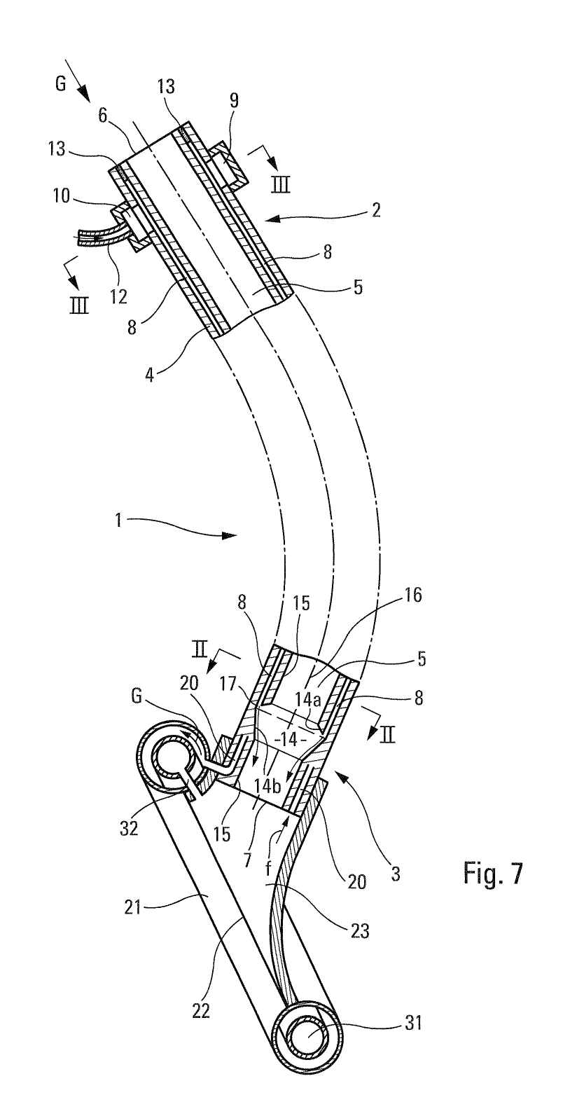

FIG. 7 is a schematic illustration, in a view similar to FIG. 1, of an alternative embodiment of said sealing means.

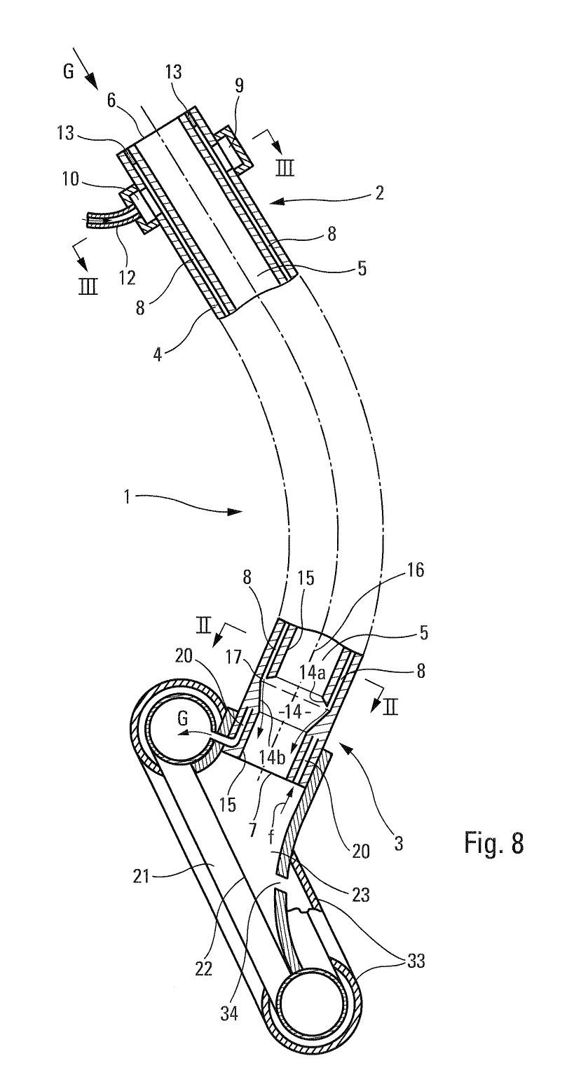

FIG. 8 is a schematic illustration, again in a view similar to FIG. 1, of another alternative embodiment of said sealing means.

FIG. 1 shows schematically, and on a large scale, only the proximal end 2 and distal end 3 of an embodiment 1 of the device according to the invention.

The device 1 comprises a flexible tube 4 (or one that is pre-shaped to adapt to the morphology of the patient) delimiting a main channel 5 that opens via the orifice 6 at the proximal end 2 and via the orifice 7 at the distal end 3.

Thus, the main channel 5 can ensure a passage between the orifices 6 and 7, of which one (the distal orifice 7) is designed to be situated inside the airways of a patient, and the other (the proximal orifice 6) is designed to be situated outside said patient. This proximal orifice 6 can open to the ambient air and, in this case, the patient can inhale fresh air and exhale the vitiated air through the main channel 5 (it would also be possible to connect the orifice 6 to a source of pressurized breathable gas and to provide a system of unidirectional valves, such that the patient inhales the breathable gas from said source by way of said main channel 5 and exhales the vitiated air into the ambient air, again by way of this main channel).

The diameter of the main channel 5 is of the order of a few millimeters. Satisfactory tests have been carried out with diameters of 3 mm, 7 mm, 8 mm and 12 mm.

Moreover, auxiliary channels 8, formed within the thickness of the wall of the tube 4, extend along almost the entire length of the main channel 5 and are designed to be connected to a source of pressurized breathable gas, as is described below.

The connection to the source of pressurized breathable gas can be effected by means of a ring 9 which surrounds the tube 4 in a leaktight manner at the proximal end 2 and which delimits a leaktight annular chamber 10 around said tube. The auxiliary channels 8 are placed in communication with the chamber 10, by virtue of local cutouts 11 in the wall of the tube 4, and said chamber 10 is connected to said source of breathable gas by a conduit 12. Of course, the proximal ends of the channels 8 are closed off, for example by plugs 13 introduced from the proximal end face of the tube 4.

The auxiliary channels 8 have a diameter smaller than that of the main channel 5. The diameter of the auxiliary channels 8 is preferably less than 1 mm and is advantageously of the order of 400 to 800 microns. At the distal end, the auxiliary channels 8 open into a recess 14 of the inner wall 15 of the tube 4. The recess 14 is annular and is centered on the axis 16 of the distal end 3. It comprises a face 14a, which is substantially transverse or slightly inclined so as to form a widening of the main channel 5 into which said auxiliary channels 8 open via their orifices 17, and a face 14b, which follows the face 14a and converges in the direction of the axis 16.

Thus, when the auxiliary channels 8 are supplied with pressurized breathable gas via the elements 9 to 12, the corresponding jets of gas strike the inclined face 14b, which deflects them in the direction of the axis (see the arrows in FIG. 1 at the outlet of the orifices 17), generating inside the distal end 3 of the main channel 5 a pressure zone of oblong shape that starts at said distal orifices 17 and continues in the direction of the distal orifice 7 along the axis 16 of said distal end 3. The cross section of this pressure zone decreases progressively from the recess 14 to the distal orifice 7, said pressure zone moving away progressively from the inner wall 15 of the tube 4 so as to occupy only the central part of the distal end 3 of the latter. Downstream of the pressure zone, the deflected jets of breathable gas generate, in the vicinity of the axis 16, a low pressure zone that promotes the flow of gas inside the main channel 5, from the proximal orifice to the distal orifice. This promotes the inhalation by the patient.

As is shown in FIGS. 2 and 3, the auxiliary channels 8 are arranged regularly about the axis of the tube 4. They may vary in number depending on use (adult or child), but are generally between three and nine in number.

The tube 4 of the device according to the invention can be made of any material already used in respiratory probes, for example a polyvinyl chloride, with an optional coating of silicone.

Supplementary channels 20 are provided within the thickness of said tube 4. These channels 20 can be used for different purposes, for example injection of a fluid medicament, pressure measurement, collection of gas samples (as is indicated symbolically by the arrow f pointing into a channel 20 in the lower part of FIG. 1), and, as will be described below, inflation of the sealing cuff. It will be noted that, in FIG. 1, for reasons of simplifying the drawing, channels 8 and portions of channels 20 have been shown, even though these channels are situated in different planes (see FIGS. 2 and 3).

As is shown in FIGS. 1, 4 and 5, an inflatable cuff 21 of annular shape is attached to the distal end 3 of the tube 4 and is carried at the periphery 22 of a component 23. The component 23 is at least approximately the shape of a trumpet and is fitted via its small end onto the distal end 3 of the tube 4. The widened end of the component 23 is slanted, in such a way that said periphery 22 and the inflatable cuff 21 supported by it are inclined with respect to the axis 16 of the distal end 3. In this way, when the device 1 is introduced in the deflated state into a patient 25 by way of the mouth 26 and the pharynx 27, the cuff 21, after inflation by an inflation gas G delivered from a source (not shown) to the device 1 and then transmitted to the cuff 21 by way of a channel 20, is able to surround the larynx 28 and ensure an at least substantially leaktight gaseous communication between the trachea 29 of the patient 25 and the main channel 5 of the device 1 (see FIG. 6). In this latter position, the cuff 21 partially obturates the esophagus 30.

It is thus possible, without introducing the tube 4 into the trachea, to ventilate the lungs (not shown) of the patient 25 with the aid of a breathable gas introduced into the main channel 5 via the channels 8 and the deflection means 14b and to allow the vitiated gas from said lungs to be exhaled to the outside (see the two arrows in FIG. 6).

FIGS. 7 and 8, in views similar to FIG. 1, show the device 1 provided with sealing means that cause little or no trauma to the mucous membranes of the pharynx 27 of the patient 25. All the elements shown in FIG. 1 and described with reference to the latter also appear in FIGS. 7 and 8.

In FIG. 7, the annular cuff 21, which can be inflated by the inflation gas G, contains another annular cuff 31 connected via a gaseous communication 32 to the inside of the component 23, that is to say to the distal end 3 of the device 1 downstream of the deflection means 14b.

In FIG. 8, by contrast, the annular cuff 21 is contained in another annular cuff 33, which is also connected to the inside of the component 23 via a gaseous communication 34.

It will be readily appreciated that, by way of the gaseous communications 32 and 34, the cuffs 31 and 33 are inflated during the injections of the breathable gas via the channels 8, whereas they are not inflated between the injections.

Consequently, the pressure of the inflation gas G can be set at a level below that necessary for the cuff 21 alone to ensure leaktightness since, during the injections, this low pressure level will be supplemented by the pressure inside the cuffs 31 or 33.

Thus, outside the periods of injection of the breathable gas, the sealing means 21, 32 and 21, 33 cause less trauma than the cuff 21 alone.

It will be noted that the respiratory assistance device according to the present invention can be designed to be lightweight and portable, so as to be able to be used in emergency situations and in first aid outside the confines of a hospital or clinic.

1. A respiratory assistance device which is tubular and which forms a main channel (5) connecting the proximal end (2) and the distal end (3) of said device (1) and of which said distal end (3) is designed to be introduced into the mouth (26) of a patient (25) in the direction of the patient's lungs, while said proximal end (2) is designed to remain outside the mouth of said patient, said device comprising:
at least one auxiliary channel (8) which opens into said main channel (5) near said distal end (3) and through which a jet of breathable gas can be injected into said main channel (5); and
deflection means (14b) by which said jet of breathable gas injected through said auxiliary channel (8) is deflected into said main channel (5),
comprising sealing means (21, 31, 33) which surround said distal end (3) and which, in the area of the pharynx (28) of said patient, are able to ensure an at least substantially leaktight gaseous communication between the trachea (29) of said patient and said main channel (5) of the device.

2. The respiratory assistance device as claimed in claim 1, wherein said sealing means comprise at least one cuff (21) of annular shape which is able to surround the larynx (27) of said patient and is inflatable under the action of an inflation gas (G) delivered to said device.

3. The respiratory assistance device as claimed in claim 2, wherein said cuff (21) is supplied with inflation gas (G) by way of at least one channel (20) formed within the thickness of the wall of said device.

4. The respiratory assistance device as claimed in claim 2, wherein said sealing means comprise two cuffs (21; 31, 33) of annular shape, of which one is arranged inside the other and which are able to surround the larynx (27) of said patient, one (21) of said cuffs being inflatable under the action of said inflation gas (G) delivered to said device (1), and the other (31, 33) being in gaseous communication with said distal end (3) of the device downstream of said deflection means (14b).

5. The respiratory assistance device as claimed in claim 4, wherein said cuff (31) in gaseous communication with the distal end (3) of the device is arranged inside said cuff (21) which can be inflated by said inflation gas.

6. The respiratory assistance device as claimed in claim 4, wherein said cuff (33) in gaseous communication with the distal end (3) of the device is arranged outside said cuff (21) which can be inflated by said inflation gas.

7. The respiratory assistance device as claimed in claim 1, wherein said sealing means (21, 31, 33) are carried at the widened and slanted periphery (22) of a trumpet-shaped component (23) that can be attached to said distal end (3) of the respiratory assistance device (1).

8. The respiratory assistance device as claimed in claim 7, wherein said trumpet-shaped component (23) is attached with a form fit to said distal end (3) of the respiratory assistance device (1).

9. The respiratory assistance device as claimed in claim 7, wherein said trumpet-shaped component (23) is integrally connected permanently to said distal end (3) of the respiratory assistance device (1).
